(19) United States
(12) Patent Application Publication
(10) Pub. No.: US 2024/0180804 A1
(43) Pub. Date: Jun. 6, 2024

(54) Mouthwash Composition

(71) Applicant: Colgate-Palmolive Company, New York, NY (US)

(72) Inventors: Changlong CHEN, New Brunswick, NJ (US); Hongwei Shen, Holmdel, NJ (US); Michael Stranick, Bridgewater, NJ (US)

(21) Appl. No.: 18/512,276

(22) Filed: Nov. 17, 2023

Related U.S. Application Data
(60) Provisional application No. 63/426,112, filed on Nov. 17, 2022.

(51) Int. Cl. A61K 8/41; A61Q 11/02

(52) U.S. Cl. CPC A61K 8/41; A61Q 11/02; A61K 2800/33; A61K 2800/74

(57) ABSTRACT

The present disclosure relates to a mouthwash composition containing an amine fluoride or amine base plus fluoride and an acyl glutamate, as well as related methods for use of said compositions.

### FIELD

The present disclosure relates to mouthwash compositions containing an amine fluoride and an acyl glutamate, as well as related methods for use of said compositions.

### BACKGROUND

Fluoride is one of the agents commonly used to combat dental caries. Fluoride can greatly help dental health by strengthening the tooth enamel, making it more resistant to tooth decay. Fluoride also helps remineralize weakened tooth enamel and reverses early signs of tooth decay. Many oral care products such as toothpastes, mouthwashes and professional dental treatment products contain fluoride. Amine fluorides are well known fluoride ion sources used in oral care products. The amine base has a hydrophobic non-polar tail and a hydrophilic polar amine head. Therefore, amine fluorides act like surfactants. Due to their surface activity, amine fluorides form a thin hydrophobic film on the tooth enamel on oral use of the hygiene composition. Thus, on the one hand, the tooth enamel becomes more resistant to acid attacks on account of the CaF₂ covering layer formed. On the other hand, the long-chain hydrocarbon residues form a hydrophobic layer which prevents the formation of deposits and the attack of the acidic degradation products on the tooth enamel.

Increasing the viscoelasticity of mouthwashes favors the film formation on the surfaces, resulting in greater retention of actives on enamel surfaces. It has been known that the viscoelasticity can be tuned by the addition of cellulosic polymer, gum polymer and poly acrylate polymer.

There is a need for mouthwash formulations containing amine fluorides with improved film forming activities on the enamel surface.

### BRIEF SUMMARY

In an aspect, the invention provides a mouthwash composition comprising an amine fluoride or amine base plus fluoride and an acyl glutamate. In certain embodiments, the amine fluoride is olaflur (N′-octadecyltrimethylenediamine-N,N,N′-tris(2-ethanol)-dihydrofluoride). In some embodiments, the composition is free or substantially free of cellulosic polymers, gum polymers and polyacrylate polymers.

In some embodiments, the amine fluoride is present in an amount of from 0.5% to 1.5%, e.g., from 0.5% to 1.2%, from 0.5% to 1%, from 0.7% to 1.5%, from 0.7% to 1.2%, from 0.7% to 1%, from 0.8% to 1.5%, from 0.8% to 1.2%, from 0.8% to 1%, from 0.85% to 0.95%, or about 0.9% by weight of the composition.

In some embodiments, the acyl glutamate is C₈₋₂₅ acyl glutamate, e.g., C₈₋₁₈ acyl glutamate, C₁₀₋₁₈ acyl glutamate, or C₁₀₋₁₆ acyl glutamate. In some embodiments, the acyl glutamate is selected from dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, and sodium undecylenoyl glutamate and mixtures thereof. In certain embodiments, the acyl glutamate is cocoyl glutamate, e.g., sodium cocoyl glutamate

In some embodiments, the acyl glutamate is present in an amount of from 0.05% to 1%, e.g., from 0.05% to 0.6%, from 0.05% to 0.5%, from 0.05% to 0.4%, from 0.05% % to 0.3%, from 0.05% % to 0.2%, from 0.05% % to 0.15%, from 0.1% to 0.6%, from 0.1% to 0.5%, from 0.1% to 0.4%, from 0.1% % to 0.3%, from 0.1% to 0.2%, from 0.1% % to 0.15%, from 0.12% to 0.16%, from 0.12% to 0.15%, from 0.13% to 0.15%, or about 0.14% by weight of the composition.

In another aspect, the invention provides a method comprising applying an effective amount of any of mouthwash compositions as disclosed herein to the oral cavity to a subject in need thereof, to (i) reduce or inhibit formation of dental caries, (ii) reduce, repair or inhibit precarious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity.

In another aspect, the invention provides the use of an acyl glutamate in a mouthwash composition comprising an amine fluoride, e.g., any of mouthwash compositions as disclosed herein for improving film forming activities of the mouthwash on the enamel surface (e.g., tooth surface).

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### BRIEF DESCRIPTION OF FIGURES

FIG. 1 shows pictures of dripping mouthwash solution from pipettes (left: Formulation B, viscosity 1.00 cP; middle: Formulation C, viscosity 2.71 cP; Right: Colgate Sensitive Pro-Relief mouthwash, viscosity 23 cP).

### DETAILED DESCRIPTION

The following description of various typical aspect(s) is merely exemplary in nature and is in no way intended to limit the disclosure, its application, or uses.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight. The amounts given are based on the active weight of the material.

In the present invention, it has been found that the use of an acyl glutamate (e.g., sodium cocoyl glutamate (SCG)) as a surfactant in a mouthwash formulation containing an amine fluoride (e.g., olaflur (N′-octadecyltrimethylenediamine-N,N,N′-tris(2-ethanol)-dihydrofluoride) results in a slight viscoelasticity of the mouthwash without using any polymer, e.g., cellulosic polymers, gum polymers and polyacrylate polymers. Without intending to be bound to any theory, the strong attractive force between oppositely charged amine fluoride (AmF) and SCG molecules is believed to effectively compress the surfactant head area and facilitate a mixed wormlike micelle formation. The mixed wormlike micelle formation may result in a slight viscoelasticity. It has been further found that the combination of amine fluoride and alkyl glutamate improves film forming on the enamel surface, thus enhancing organic retention on the enamel, compared to amine fluoride/PEG-40 hydrogenated castor oil (two key ingredients in a commercial product, Elmex mouthwash). It is believed that the higher organic retention may be due to the wormlike structure of the AmF-SCG complex. Thus, the amine fluoride/acyl glutamate combination is an improved alternative to amine fluoride/PEG-40 hydrogenated castor oil in mouthwash formulations to improve film forming activities of the mouthwash on the enamel surface (e.g., tooth surface) and enhance organic retention on the enamel surface.

The present invention provides, in an aspect, a mouthwash composition (Composition 1.0) comprising an amine fluoride or amine base plus fluoride and an acyl glutamate.

For example, the invention includes:
1.1. Composition 1.0, wherein the acyl glutamate is C₈₋₂₅ acyl glutamate, e.g., C₈₋₁₈ acyl glutamate, C₁₀₋₁₈ acyl glutamate, or C₁₀₋₁₆ acyl glutamate.
1.2. Any of the preceding compositions, wherein the acyl glutamate is selected from dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, and sodium undecylenoyl glutamate and mixtures thereof.
1.3. Any of the preceding compositions, wherein the acyl glutamate is cocoyl glutamate, e.g., sodium cocoyl glutamate.
1.4. Any of the preceding compositions, wherein the acyl glutamate is present in an amount of from 0.05% to 1%, e.g., from 0.05% to 0.6%, from 0.05% to 0.5%, from 0.05% to 0.4%, from 0.05% % to 0.3%, from 0.05% % to 0.2%, from 0.05% % to 0.15%, from 0.1% to 0.6%, from 0.1% to 0.5%, from 0.1% to 0.4%, from 0.1% % to 0.3%, from 0.1% to 0.2%, from 0.1% % to 0.15%, from 0.12% to 0.15%, from 0.12% to 0.16%, from 0.13% to 0.15%, or about 0.14% by weight of the composition.
1.5. Any of the preceding compositions, wherein the amine base is a primary amine, secondary amine, tertiary amine or a combination thereof.
1.6. Any of the preceding compositions, wherein the amine base comprises or consists of a primary amine base.
1.7. Any of the preceding compositions, wherein the amine base comprises or consists of a secondary amine base.
1.8. Any of the preceding compositions, wherein the amine base comprises or consists of a tertiary amine base.
1.9. Any of the preceding compositions, wherein the amine base is plant-derived.
1.10. Any of the preceding compositions, wherein the amine base is animal-derived.
1.11. Any of the preceding compositions, wherein the amine base is derived from bovine tallow.
1.12. Any of the preceding compositions, wherein the amine base is derived from rapeseed oil or from rice bran oil.
1.13. Any of the preceding compositions, wherein the amine base is a linear or branched fatty amine or polyamine, or mixtures thereof.
1.14. The preceding composition, wherein the amine base is a saturated or unsaturated C₁₂₋₂₀ alkyl amine base or a saturated or unsaturated C₁₂₋₂₀ alkyl polyamine base, or mixtures thereof.
1.15. Any of the preceding compositions, wherein the amine base is a myristyl, palmityl, linoleyl, oleyl, or stearyl amine or polyamine, or combinations thereof.
1.16. Any of the preceding compositions, wherein the amine base is a polyamine (e.g., a monoamine base, a diamine base and/or a triamine base).
1.17. Any of the preceding compositions, wherein the amine base is a monoamine base.
1.18. Any of the preceding compositions, wherein the amine base is a diamine base.
1.19. Any of the preceding compositions, wherein the amine base is a triamine base.
1.20. Any of the preceding compositions, wherein the amine base comprises one or more of N′-octadecyltrimethylendiamine-N,N,N′-tris(2-ethanol), and/or oleyldiamine ethoxylate and/or N-octadeca-9-enylamine, and/or alkyl trihydroxyethyl propylenediamine.
1.21. Any of the preceding compositions, wherein the amine base is N′-octadecyltrimethylendiamine-N, N,N′-tris(2-ethanol).
1.22. Any of the preceding compositions, wherein the amine base is N-octadeca-9-enylamine.
1.23. Any of the preceding compositions, wherein the amine base and fluoride form amine fluoride in situ.
1.24. Any of the preceding compositions, wherein the amine fluoride is olaflur (N′-octadecyltrimethylenediamine-N, N,N′-tris(2-ethanol)-dihydrofluoride).
1.25. Any of the preceding compositions, wherein the amine fluoride is present in an amount of from 0.5% to 1.5%, e.g., from 0.5% to 1.2%, from 0.5% to 1%, from 0.7% to 1.5%, from 0.7% to 1.2%, from 0.7% to 1%, from 0.8% to 1.5%, from 0.8% to 1.2%, from 0.8% to 1%, from 0.85% to 0.95%, or about 0.9% by weight of the composition.
1.26. Any of the preceding compositions, wherein the composition comprises olaflur and cocoyl glutamate, e.g., sodium cocoyl glutamate.
1.27. Any of the preceding compositions, wherein the composition comprises olaflur in an amount of from 0.5% to 1.5% by weight of the composition and cocoyl glutamate, e.g., sodium cocoyl glutamate, in an amount of from 0.05% to 1% by weight of the composition.
1.28. Any of the preceding compositions, wherein the composition comprises olaflur in an amount of from 0.8% to 1.2% by weight of the composition and cocoyl glutamate, e.g., sodium cocoyl glutamate, in an amount of from 0.1% to 0.2% by weight of the composition.
1.29. Any of the preceding compositions, wherein the composition comprises olaflur in an amount of from 0.8% to 1%, e.g., about 0.9% by weight of the composition and cocoyl glutamate, e.g., sodium cocoyl glutamate, in an amount of from 0.12% to 0.16%, e.g., about 0.14% by weight of the composition.
1.30. Any of the preceding compositions, wherein the composition is free or substantially free of sodium lauryl sulfate.
1.31. Any of the preceding compositions, wherein the composition is free or substantially free of alkyl sulfate salts.
1.32. Any of the preceding compositions, wherein the composition is free or substantially free of anionic surfactants other than the acyl glutamate.
1.33. Any of the preceding compositions, wherein the composition does not comprise any surfactant other than the acyl glutamate and amine fluoride.
1.34. Any of the preceding compositions, wherein the composition is free or substantially free of cellulosic polymers, gum polymers and polyacrylate polymers.
1.35. Any of the preceding compositions comprising a humectant, e.g., selected from glycerin, sorbitol, propylene glycol, polyethylene glycol, xylitol, and mixtures thereof.
1.36. Any of the preceding compositions, wherein the composition comprises one or more soluble phosphate salts, e.g., selected from tetrasodium pyrophosphate (TSPP), sodium tripolyphosphate (STPP) and a combination thereof.
1.37. Any of the preceding compositions, wherein the composition comprises a basic amino acid in free or salt form, optionally wherein the basic amino acid comprises arginine.
1.38. Any of the preceding compositions, wherein the composition comprises a zinc ion source.
1.39. Composition 1.38, wherein the zinc ion source is selected from the group consisting of zinc oxide, zinc sulfate, zinc chloride, zinc citrate, zinc lactate, zinc gluconate, zinc malate, zinc tartrate, zinc carbonate, zinc phosphate and a combination thereof.
1.40. Any of Composition 1.38 to 1.39, wherein the zinc ion source is selected from the group consisting of zinc oxide, zinc citrate, and a combination thereof, optionally wherein the zinc ion source is a combination of zinc oxide and zinc citrate.
1.41. Any of the preceding compositions comprising a sweetener, optionally wherein the sweetener is saccharin.
1.42. Any of the preceding compositions comprising a breath freshener.
1.43. Any of the preceding compositions comprising a fragrance.
1.44. Any of the preceding compositions comprising a flavorant.
1.45. Any of the preceding compositions comprising a pigment.
1.46. Any of the preceding compositions comprising a preservative.
1.47. Any of the preceding compositions, wherein the composition comprises water.
1.48. Any of the preceding compositions which is free or substantially free of ethanol, e.g., having less than 0.5% ethanol by weight.
1.49. Any of the preceding compositions, wherein the mouthwash is a micellar mouthwash.

The mouthwash composition of the present invention comprises an amine fluoride or amine base plus fluoride. As used herein, the term “amine base” may refer to a primary amine base, a secondary amine base or a tertiary amine base. “Primary amine base” refers to a compound containing at least one amine in which the nitrogen atom is directly bonded to one carbon of any hybridization, except for carbonyl group carbons. “Secondary amine base” refers to a compound containing at least one amine in which the nitrogen atom is directly bonded to two carbons of any hybridization, except for carbonyl group carbons. “Tertiary amine base” refers to a compound containing at least one amine in which the nitrogen atom is directly bonded to three carbons of any hybridization, except for carbonyl group carbons. “Amine base” may be used to refer to compounds containing a plurality of primary, secondary and/or tertiary amine groups (e.g., a tertiary polyamine). In particular, the term “amine base” excludes acid addition salts (e.g., hydrochloride salts and hydrofluoride salts), and thus refers to the free base form of the molecule. In some embodiments, the amine base is selected from N′-octadecyltrimethylendiamine-N,N,N′-tris (2-ethanol), oleyldiamine ethoxylate, N-octadeca-9-enylamine, alkyl trihydroxyethyl propylenediamine, and a combination thereof. Hydrofluoride derivatives of amines are referred to herein as “amine fluorides.” In methods for the production or manufacture of a composition containing an amine fluoride, an amine base may be a precursor to forming the amine fluoride. In certain embodiments, the amine fluoride is olaflur (N′-octadecyltrimethylenediamine-N,N,N′-tris (2-ethanol)-dihydrofluoride).

In some embodiments, the amine base and fluoride form amine fluoride in situ. As used herein, the term “in situ” is used to refer to the formation of a chemical product (e.g., amine fluoride) in the mouthwash composition. For example, the reaction may be a salination reaction carried out by mixing an amine with a fluoride source and an acid, thus creating an amine fluoride and a salt.

In some embodiments, the amine fluoride is present in an amount of from 0.5% to 1.5%, e.g., from 0.5% to 1.2%, from 0.5% to 1%, from 0.7% to 1.5%, from 0.7% to 1.2%, from 0.7% to 1%, from 0.8% to 1.5%, from 0.8% to 1.2%, from 0.8% to 1%, from 0.85% to 0.95%, or about 0.9% by weight of the composition.

The mouthwash composition of the present invention comprises an acyl glutamate. Acyl glutamate is a compound in which an acyl group is linked to glutamate by an amide bond. Acyl refers to a moiety containing a double-bonded oxygen group and an alkyl group (R—C═O). As used herein, the term “acyl glutamate” may be or include free form or any acceptable salts thereof. The salt may be sodium or potassium. For example, the structure of sodium cocoyl glutamate is shown below:

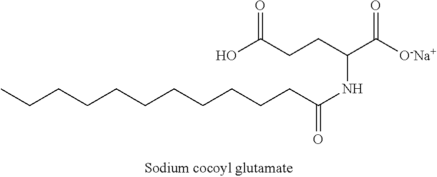

Acyl glutamates which can be used in the composition may be C₈₋₂₅ acyl glutamate, e.g., C₈₋₁₈ acyl glutamate, C₁₀₋₁₈ acyl glutamate, or C₁₀₋₁₆ acyl glutamate. The acyl group may be branched or unbranched. In some embodiments, the acyl group is unbranched. The acyl group may be saturated or unsaturated. In some embodiments, the acyl group is saturated. In some embodiments, the acyl glutamate is selected from dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, and sodium undecylenoyl glutamate and a combination thereof. In some embodiments, the acyl glutamate is cocoyl glutamate, e.g., sodium cocoyl glutamate.

In some embodiments, the acyl glutamate is present in an amount of from 0.05% to 1%, e.g., from 0.05% to 0.6%, from 0.05% to 0.5%, from 0.05% to 0.4%, from 0.05% % to 0.3%, from 0.05% % to 0.2%, from 0.05% % to 0.15%, from 0.1% to 0.6%, from 0.1% to 0.5%, from 0.1% to 0.4%, from 0.1% % to 0.3%, from 0.1% to 0.2%, from 0.1% % to 0.15%, from 0.12% to 0.16%, from 0.12% to 0.15%from 0.13% to 0.15%, or about 0.14% by weight of the composition.

In some embodiments, the mouthwash composition of the present invention is free or substantially free of sodium lauryl sulfate. In some embodiments, the composition is free or substantially free of alkyl sulfate salts. In some embodiments, the composition is free or substantially free of anionic surfactants other than the acyl glutamate. In some embodiments, the composition does not comprise any surfactant other than the acyl glutamate and amine fluoride.

In some embodiments, the composition is free or substantially free of cellulosic polymers, gum polymers and polyacrylate polymers.

The mouthwash composition of the present invention comprises water. Water employed in the preparation of the mouthwash composition should be deionized and free of organic impurities. Water may make up the balance of the mouthwash composition. In some embodiments, water is present in an amount of from 70% to 99%, e.g., 80% to 99%, 90% to 99%, by weight of the composition. This amount of water includes the free water which is added plus that amount which is introduced with other components of the oral care composition, such as with sorbitol.

The mouthwash composition of the present invention may include one or more humectants. Humectants can reduce evaporation and also contribute towards preservation by lowering water activity and can also impart desirable sweetness or flavor to compositions. Illustrative humectants may be or include, but are not limited to, glycerin, propylene glycol, polyethylene glycol, sorbitol, xylitol, or the like, or any mixture or combination thereof. In a preferred embodiment, the orally acceptable vehicle may be or include, but is not limited to, glycerin or sorbitol. In some embodiments, the humectant is selected from glycerin, sorbitol and a combination thereof.

The mouthwash composition of the present invention may include a basic amino acid in free or salt form. The basic amino acids which can be used in the compositions include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrullene, ornithine, creatine, histidine, diaminobutanoic acid, diaminopropionic acid, salts thereof or combinations thereof. In a particular embodiment, the basic amino acids are selected from arginine, lysine, citrullene, and ornithine. The basic amino acids of the oral care composition may generally be present in the L-form or L-configuration. The basic amino acids may be provided as a salt of a di- or tri-peptide including the amino acid. In some embodiments, at least a portion of the basic amino acid present in the mouthwash composition is in the salt form. In some embodiments, the basic amino acid is arginine, for example, L-arginine, or a salt thereof. Arginine may be provided as free arginine or a salt thereof. For example, Arginine may be provided as arginine phosphate, arginine hydrochloride, arginine sulfate, arginine bicarbonate, or the like, and mixtures or combinations thereof. The basic amino acid may be provided as a solution or a solid. For example, the basic amino acid may be provided as an aqueous solution. In some embodiment, the amino acid includes or is provided by an arginine bicarbonate solution. For example, the amino acid may be provided by an about 40% solution of the basic amino acid, such as arginine bicarbonate or alternatively called as arginine carbamate.

The mouthwash composition of the present invention may include a zinc ion source. The zinc ion source may be or include a zinc ion and/or one or more zinc salts. For example, the zinc salts may at least partially dissociate in an aqueous solution to produce zinc ions. Illustrative zinc salts may include, but are not limited to, zinc lactate, zinc oxide, zinc chloride, zinc phosphate, zinc citrate, zinc acetate, zinc borate, zinc butyrate, zinc carbonate, zinc formate, zinc gluconate, zinc glycerate, zinc glycolate, zinc picolinate, zinc proprionate, zinc salicylate, zinc silicate, zinc stearate, zinc tartrate, zinc undecylenate, and mixtures thereof. In some embodiments, the zinc ion source is selected from zinc oxide, zinc citrate, and a combination thereof.

The mouthwash composition of the present invention may include a preservative. Suitable preservatives include, but are not limited to, sodium benzoate, potassium sorbate, methylisothiazolinone, paraben preservatives, for example methyl p-hydroxybenzoate, propyl p-hydroxybenzoate, and mixtures thereof. In certain embodiments, the preservative is sodium benzoate.

The mouthwash composition of the present invention may include a sweetener such as, for example, saccharin, for example sodium saccharin, acesulfam, neotame, cyclamate or sucralose; natural high-intensity sweeteners such as thaumatin, stevioside or glycyrrhizin; or such as sorbitol, xylitol, maltitol or mannitol. In certain embodiments, the sweetener is saccharin, e.g., sodium saccharin.

The mouthwash composition of the present invention may include a flavoring agent. Suitable flavoring agents include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole.

The mouthwash composition of the present invention may include one or more pH modifying agents. For example, the oral care composition may include one or more acidifying agents and/or one or more basifying agents configured to reduce and/or increase the pH thereof, respectively. Illustrative acidifying agents and/or one or more basifying agents may be or include, but are not limited to, an alkali metal hydroxide, such as sodium hydroxide and/or potassium hydroxide, citric acid, hydrochloric acid, or the like, or combinations thereof.

The mouthwash composition of the present invention may also include one or more buffering agents configured to control or modulate the pH within a predetermined or desired range. Illustrative buffering agents may include, but are not limited to, sodium bicarbonate, sodium phosphate, sodium carbonate, sodium acid pyrophosphate, sodium citrate, and mixtures thereof. Sodium phosphate may include monosodium phosphate (NaH₂PO₄), disodium phosphate (Na₂HPO₄), trisodium phosphate (Na₃PO₄), and mixtures thereof. In a typical embodiment, the buffering agent may be anhydrous sodium phosphate dibasic or disodium phosphate and/or sodium phosphate monobasic. In another embodiment, the buffering agent includes anhydrous sodium phosphate dibasic or disodium phosphate, and phosphoric acid (e.g., syrupy phosphoric acid; 85%-Food Grade).

The mouthwash composition of the present invention may include anticalculus agents. Illustrative anticalculus agents may include, but are not limited to, phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates. In some embodiments, the anticalculus agent includes tetrasodium pyrophosphate (TSPP), sodium tripolyphosphate (STPP), or a combination thereof.

The mouthwash composition of the present invention may include an antioxidant. Any orally acceptable antioxidant may be used, including, but not limited to, butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, or the like, or combinations and mixtures thereof.

All ingredients for use in the compositions described herein should be orally acceptable. As used herein, “orally acceptable” may refer any ingredient that is present in a composition as described in an amount and form which does not render the composition unsafe for use in the oral cavity.

In another aspect, the present invention provides a method to (i) reduce or inhibit formation of dental caries, (ii) reduce, repair or inhibit pre-carious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity, comprising applying an effective amount of any of mouthwash compositions as disclosed herein to the oral cavity of a subject in need thereof.

In another aspect, the invention provides a method comprising applying an effective amount of any of mouthwash compositions as disclosed herein to the oral cavity to a subject in need thereof, to (i) reduce or inhibit formation of dental caries, (ii) reduce, repair or inhibit precarious lesions of the enamel, (iii) reduce or inhibit demineralization and promote remineralization of the teeth, (iv) reduce hypersensitivity of the teeth, (v) reduce or inhibit gingivitis, (vi) promote healing of sores or cuts in the oral cavity, (vii) reduce levels of acid producing bacteria, (viii) reduce or inhibit microbial biofilm formation in the oral cavity, (ix) reduce or inhibit plaque formation in the oral cavity, (x) promote systemic health, or (xi) clean teeth and oral cavity.

In another aspect, the invention provides the use of an acyl glutamate in a mouthwash composition comprising an amine fluoride, e.g., any of mouthwash compositions as disclosed herein for improving film forming activities of the mouthwash on the enamel surface (e.g., tooth surface). In some embodiments, the composition is free or substantially free of cellulosic polymers, gum polymers and polyacrylate polymers.

### EXAMPLES

Three solutions having the formulas as indicated in Table 1 are prepared. Formulation A is a solution of amine fluoride (Olaflur). Formulation B contains an amine fluoride (Olaflur) and PEG-40 hydrogenated castor oil, which is used as a surfactant in a commercial mouthwash (Elemex) formulation, while Formulation C contains Olaflur and sodium cocoyl glutamate (SCG). The Elemex mouthwash contains 0.89% Olaflur and 0.27% PEG-40 hydrogenated castor oil.

TABLE 1

| | Formu- | Formu- | Formu- |
|---|---|---|---|
| | lation A | lation B | lation C |
| Amine fluoride (Olaflur) | 0.9% | 0.9% | 0.9% |
| PEG-40 hydrogenated castor oil (90%) | | 0.3% | |
| Sodium cocoyl glutamate (42%) | | | 0.33% |
| water | q.s. | q.s. | q.s. |

The viscosity of Formulations A-C is measured with an Ubbelohde viscometer. The results are shown in Table 2.

TABLE 2

| Formulation | Viscosity (cP) |
|---|---|
| A | 1.00 |
| B | 1.00 |
| C | 2.71 |

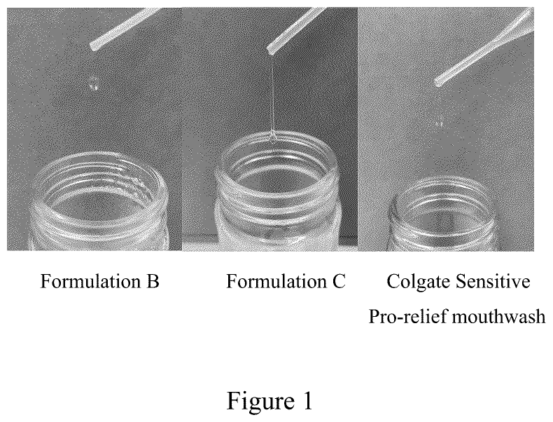

Both Formulations A and B have a viscosity of 1.00 cP, close to that of pure water. However, Formulation C has a higher viscosity, around 2.71 cP, but still within the viscosity range of regular mouthwash products. It is believed that strong interaction between oppositely charged amine fluoride (AmF) and SCG molecules facilitates the cylindrical micelle formation, thus resulting in the viscoelasticity. FIG. 1 shows pictures of dripping mouthwash solutions from pipettes. In FIG. 1, the viscoelasticity of formulation C is evidenced by the formation of a liquid filament while dripping the solution from a pipette (FIG. 1, middle). In comparison, Formulation B drips as a typical water droplet (FIG. 1, left). The viscoelasticity is not directly related to the apparent viscosity. Colgate Sensitive Pro-Relief mouthwash, which has a viscosity of about 23 cP, drips as a water droplet without filament formation as well (FIG. 1, right).

Next, the active retention is examined on bovine enamel blocks. Before treatment, each block is gently cleaned using a polish pad and then prewashed with 5 ml DI water for 5 minutes to remove surface residues. The block was then etched in 1% citric acid solution at pH of 3.5 for 2 minutes followed by repeated DI water washing. After draining the excess water on the surface, the baseline of the enamel block is taken by XPS. Then, each block is soaked in 2 mL mouthwash solution for 30 seconds, followed by transferring to a weighing boat to drain the excess liquid and air dry. The organic retention on the enamel block is then analyzed by X-Ray photoelectron spectroscopy (XPS). Duplicates are run for each formulation. The organic retention results are shown in Table 3.

TABLE 3
Organic retention on bovine enamel surfaces measured by XPS

Atomic Percent

| Sample | | Cₜₒₜₐₗ | CH₂ | Nₜₒₜₐₗ | N | N⁺ |
|---|---|---|---|---|---|---|
| Untreated | mean | 18.23 | 10.82 | 0.79 | 0.79 | — |

0.135% AmF
(Formulation A)

| | | | | | | |
|---|---|---|---|---|---|---|
| Sample1 | | 16.71 | 11.72 | 0.84 | 0.71 | 0.12 |
| Sample2 | | 28.51 | 21.42 | 1.63 | 1.21 | 0.42 |
| | mean | 22.61 | 16.57 | 1.24 | 0.96 | 0.27 |

0.135% AmF/PEG-40
(Formulation B)

| | | | | | | |
|---|---|---|---|---|---|---|
| Sample3 | | 28.48 | 21.73 | 1.35 | 1.01 | 0.34 |
| Sample4 | | 32.07 | 24.03 | 1.53 | 1.20 | 0.36 |
| | mean | 30.28 | 22.88 | 1.44 | 1.11 | 0.35 |

0.135% AmF/Na cocoyl glutamate
(Formulation C)

| | | | | | | |
|---|---|---|---|---|---|---|
| Sample5 | | 38.98 | 31.12 | 1.89 | 1.51 | 0.35 |
| Sample6 | | 38.36 | 29.86 | 2.27 | 1.92 | 0.35 |
| | mean | 38.67 | 30.49 | 2.08 | 1.72 | 0.35 |

The total carbon retention Ctotal is highest in Formulation C among the tested formulations: Formulation C (38.67%)>Formulation B (30.28%)>Formulation A (22.61%). The total nitrogen retention Ntotal is also highest in Formulation C among the tested formulations: Formulation C (2.08%)>Formulation B (1.44%)>Formulation A (1.24%). Viscoelastic Formulation C treated enamel exhibits higher CH₂ and N levels than the other treated enamel samples. Compared to Formulation B (simple solution of current Elmex mouthwash), Ctotal is increased by 27.7% and Ntotal is increased by 44.4% in Formulation C, suggesting higher deposition of the actives and surfactants on the enamel in Formulation C. It is believed that the higher organic retention in Formulation C is likely due to the wormlike structure of the AmF-SCG complex. With a higher viscosity and slight viscoelasticity, formulation C may form a film on the enamel surface, thus resulting in higher organic retention.

While the present disclosure has been described with reference to embodiments, it will be understood by those skilled in the art that various modifications and variations may be made therein without departing from the scope of the present disclosure as defined by the appended claims.

1. A mouthwash composition comprising an amine fluoride or amine base plus fluoride and an acyl glutamate.

2. The composition according to claim 1, wherein the acyl glutamate is C₈₋₂₅ acyl glutamate.

3. The composition according to claim 1, wherein the acyl glutamate is selected from dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, and sodium undecylenoyl glutamate and mixtures thereof.

4. The composition according to claim 1, wherein the acyl glutamate is cocoyl glutamate, e.g., sodium cocoyl glutamate.

5. The composition according to claim 1, wherein the acyl glutamate is present in an amount of from 0.05% to 1% by weight of the composition.

6. The composition according to claim 1, wherein the acyl glutamate is present in an amount of from 0.12% to 0.16% by weight of the composition.

7. The composition according to claim 1, wherein the amine base is a linear or branched fatty amine or polyamine.

8. The composition according to claim 1, wherein the amine base is a saturated or unsaturated C₁₂₋₂₀ alkyl amine base or a saturated or unsaturated C₁₂₋₂₀ alkyl polyamine base.

9. The composition according to claim 1, wherein the amine base is a myristyl, palmityl, linoleyl, oleyl, or stearyl amine or polyamine, or N′-octadecyltrimethylendiamine-N, N,N′-tris(2-ethanol), or N-octadeca-9-enylamine, and combinations thereof.

10. The composition according to claim 1, wherein the amine base and fluoride form amine fluoride in situ.

11. The composition according to claim 1, wherein the amine fluoride is olaflur (N′-octadecyltrimethylenediamine-N,N,N′-tris(2-ethanol)-dihydrofluoride).

12. The composition according to claim 1, wherein the amine fluoride is present in an amount of from 0.5% to 1.5% by weight of the composition.

13. The composition according to claim 1, wherein the amine fluoride is present in an amount of from 0.8% to 1.2% by weight of the composition.

14. The composition according to claim 1, wherein the composition comprises olaflur in an amount of from 0.8% to 1.2% by weight of the composition and cocoyl glutamate, e.g., sodium cocoyl glutamate in an amount of from 0.12% to 0.16% by weight of the composition.

15. The composition according to claim 1, wherein the composition is free or substantially free of sodium lauryl sulfate.

16. The composition according to claim 1, wherein the composition is free or substantially free of anionic surfactants other than the acyl glutamate.

16. The composition according to claim 1, wherein the composition is free or substantially free of cellulosic polymers, gum polymers and polyacrylate polymers.

17. Canceled.

18. A method to:
(i) reduce or inhibit formation of dental caries;
(ii) reduce, repair or inhibit pre-carious lesions of the enamel;
(iii) reduce or inhibit demineralization and promote remineralization of the teeth;
(iv) reduce hypersensitivity of the teeth;
(v) reduce or inhibit gingivitis;
(vi) promote healing of sores or cuts in the oral cavity;
(vii) reduce levels of acid producing bacteria;
(viii) reduce or inhibit microbial biofilm formation in the oral cavity;
(ix) reduce or inhibit plaque formation in the oral cavity;
(x) promote systemic health; or (xi) clean teeth and oral cavity;
comprising applying an effective amount of a composition according to claim 1 to the oral cavity to a subject in need thereof.
